(19) United States
(12) Patent Application Publication
(10) Pub. No.: US 2013/0243826 A1
(43) Pub. Date: Sep. 19, 2013

(54) TOPICAL COMPOSITIONS FOR USE IN THE CONTROL OF SEXUALLY TRANSMITTED DISEASES

(71) Applicant: Robert Martyn, Ontario (CA)

(72) Inventor: Robert Martyn, Ontario (CA)

(21) Appl. No.: 13/801,993

(22) Filed: Mar. 13, 2013

(30) Foreign Application Priority Data
Mar. 14, 2012 (ZA) 2012/01870

(51) Int. Cl. A61K 33/30

(52) U.S. Cl. CPC A61K 33/30
USPC 424/400; 424/642

(57) ABSTRACT

Topical compositions for use in the control of sexually transmitted diseases (for example HIV/AIDS) are provided. The compositions include zinc compounds, for example zinc oxide. The zinc compounds are non water-soluble or sparingly water soluble and may be present in amounts between 25% and 50% by weight of the compositions. In selected embodiments of the invention, from 1% to 60% inclusive by weight of the compositions comprises zinc compounds having a particle size not exceeding 100 nanometers. The compositions further include pharmaceutically acceptable carriers within which the zinc compounds are dispersed. A method of manufacturing a topical composition for prophylaxis against sexually transmitted diseases is also provided, the method including heating a carrier to a liquid form and mixing with it a zinc compound as described above. Also described are mechanical contraceptive devices, for example condoms, coated with topical compositions according to the invention.

### FIELD OF THE INVENTION

THIS INVENTION relates to topical compositions for use in the control of sexually transmitted diseases.

Sexually transmitted diseases (“STDs”), for example, the Human Immunodeficiency Virus (“HIV”), Herpes and the Human Papillomavirus (“HPV”), are a significant global health problem. Condoms are partially effective in protecting against STDs but there are factors which can limit the appeal and effectiveness of condoms. For example, there is a tendency to forgo condom use because of a perceived reduction of sensation associated with condoms. Even where condoms are used carefully, breakages can occur, leaving users exposed to potentially risky situations in which STDs may be contracted. A need exists for additional products suitable for controlling STDs.

U.S. Pat. No. 8,062,631 teaches a preferred formulation of a cream consisting of from 1% to 8% by weight concentration of zinc oxide in combination with carrier agents including lanolin, alcohol, silicone and other components.

U.S. Pat. Nos. 5,624,675 and 5,482,053 teach formulations to be applied to a man's penis and genitals, with and without the use of a condom, to prevent the transmission of STDs such as HIV. The formulations include water soluble zinc salts. Zinc oxide (which is only sparingly soluble in water) is not disclosed as a possible component of the formulations.

Through inventive analysis and reasoning the Inventor has identified certain disadvantages associated with formulations in the prior art. One disadvantage arises from the unsightly colours that such formulations sometimes have, especially when used by persons whose skin-tone contrasts with such colours. Other disadvantages relate to inadequate microbicidal activity and inadequate physical barrier properties. It is an object of the present invention to address, at least in part, these disadvantages.

### SUMMARY OF THE INVENTION

According to a first aspect of the invention there is provided a topical composition for use in the control of STDs, wherein:

between 25% and 50% by weight of said composition is comprised of at least one pharmaceutically acceptable zinc compound selected from the group consisting of non water-soluble and sparingly water-soluble zinc compounds; and

said composition further includes at least one pharmaceutically acceptable carrier within which the at least one zinc compound is dispersed.

Also provided is a topical composition for use in the control of STDs, wherein:

from 1% to 60% inclusive by weight of said composition is comprised of at least one pharmaceutically acceptable zinc compound selected from the group consisting of non water-soluble and sparingly water-soluble zinc compounds having particles the majority of which have a maximum dimension not exceeding 100 nanometers; and

said composition further includes at least one pharmaceutically acceptable carrier within which the at least one zinc compound is dispersed.

For purposes of the present specification the word “dispersed” is intended to be construed broadly and includes reference to dissolved, mixed and otherwise distributed material.

In a particularly preferred embodiment, the zinc compound is present in the topical composition in a ratio or concentration of approximately 40% by weight.

The zinc compound may be selected from the group consisting of zinc oxide and zinc salts which are insoluble or sparingly soluble in water. In a preferred embodiment of the invention the zinc compound is zinc oxide.

For purposes of the present specification, the phrase “by weight”, used in connection with the ratio or concentration of an ingredient or compound, refers to the percentage of the compound on a weight-by-weight (w/w) basis, and indicates the percentage ratio of the compound itself (e.g. zinc oxide), not the percentage ratio of any elemental components thereof (e.g. elemental zinc) released by decomposition, ionization, or the like, of the compound.

The topical composition may include a barrier substance. The barrier substance may be present in a ratio or concentration of from 1% to 99% by weight, preferably from 1% to 30% by weight. The barrier substance may include a semi-solid substance, for example a hydrocarbon base or mixture such as petrolatum, otherwise known as petroleum jelly, mineral oil jelly or soft paraffin.

The topical composition may include components selected from the group consisting of emulsifying bases or agents, absorption bases, skin-conditioning and/or protecting agents, emollients or moisturizers, humectants, surfactants, microbicides (and compounds whose decomposition products are microbicides) and preservatives.

The topical composition may include a colourant. The colourant may be adapted to impart to the composition a colour selected to match a predetermined human skin-tone.

The topical composition may preferably, although not necessarily, include components selected from the group consisting of the following, present in the ranges stated (by weight):

| | |
|---|---|
| zinc oxide or sparingly water soluble zinc salts | between 25% and 50%; |
| petrolatum | 1% to 30%; |
| lanolin oil | 1% to 20%; |
| mineral oil | 1% to 20%; |
| emulsifying agent (e.g. emulsifying wax) | 5% to 15%; |
| beeswax | 2% to 8%; |
| glyceryl caprylate | 1% to 2%; |
| glyceryl laurate | 0.1% to 2%; |
| dioxide and/or diol (for example, phenoxy- | 0.1% to 2%; and |
| ethanol, caprylyl glycol, hexylene glycol and | |
| mixtures of these) | |
| iron oxide | 0.1% to 2%. |

According to a further aspect of the invention there is provided a pharmaceutical formulation wherein:

between 25% and 50% by weight of said formulation is comprised of at least one pharmaceutically acceptable zinc compound selected from the group consisting of non water-soluble and sparingly water-soluble zinc compounds; and

said formulation further includes at least one pharmaceutically acceptable carrier within which the at least one zinc compound is dispersed.

Also provided is a pharmaceutical formulation wherein:

from 1% to 60% inclusive by weight of said formulation is comprised of at least one pharmaceutically acceptable zinc compound selected from the group consisting of non water-soluble and sparingly water-soluble zinc compounds having particles the majority of which have a maximum dimension not exceeding 100 nanometers; and

said formulation further includes at least one pharmaceutically acceptable carrier within which the at least one zinc compound is dispersed.

The zinc compound may be as hereinbefore described. The formulation may include a barrier substance and other components as hereinbefore described, in the ratios or concentrations as hereinbefore described.

In yet a further aspect, the invention provides a kit for use in prophylaxis against STDs, which includes

a dispensing container containing a topical composition as hereinbefore described; and

a set of instructions for applying said topical composition to a genital surface.

For purposes of this specification the term “genital surface” is intended to be interpreted broadly, and shall include reference to at least one surface of a mechanical prophylactic or contraceptive device. For example, the term shall include reference to a surface of a prophylactic or contraceptive device such as a male or female condom.

In other words, the topical composition, pharmaceutical formulation and kit described herein may be used in combination with contraceptive devices, for example, male or female condoms.

In a further aspect, the invention provides a method of prophylaxis against STDs which includes applying a topical composition as described herein to at least one genital surface.

In yet another aspect the invention provides the use, in the manufacture of a topical composition for prophylaxis against STDs, of the following:

at least one pharmaceutically acceptable zinc compound selected from the group consisting of non water-soluble and sparingly water-soluble zinc compounds, in an amount calculated to result in a ratio of the at least one zinc compound, in the manufactured composition, ranging between 25% and 50% by weight; and

at least one pharmaceutically acceptable carrier component suitable for dispersal within it of the at least one zinc compound during said manufacture.

Also provided is the use, in the manufacture of a topical composition for prophylaxis against STDs, of the following:

at least one pharmaceutically acceptable zinc compound selected from the group consisting of non water-soluble and sparingly water-soluble zinc compounds having particles the majority of which have a maximum dimension not exceeding 100 nanometers, in an amount calculated to result in a ratio of the at least one zinc compound, in the manufactured composition, ranging from 1% to 60% inclusive by weight of said composition; and

at least one pharmaceutically acceptable carrier component suitable for dispersal within it of the at least one zinc compound during said manufacture.

The zinc compound may be as hereinbefore described.

The invention also provides a method of manufacturing a topical composition for prophylaxis against STDs, which includes the steps of

heating at least one pharmaceutically acceptable carrier; and

mixing with said carrier at least one pharmaceutically acceptable zinc compound selected from the group consisting of non water-soluble and sparingly water-soluble zinc compounds, in an amount calculated to result in a ratio of the at least one zinc compound, in the manufactured composition, ranging between 25% and 50% by weight.

Also provided is a method of manufacturing a topical composition for prophylaxis against STDs, which includes the steps of

heating at least one pharmaceutically acceptable carrier; and

mixing with said carrier at least one pharmaceutically acceptable zinc compound selected from the group consisting of non water-soluble and sparingly water-soluble zinc compounds having particles the majority of which have a maximum dimension not exceeding 100 nanometers, in an amount calculated to result in a ratio of the at least one zinc compound, in the manufactured composition, ranging from 1% to 60% inclusive by weight of said composition.

The zinc compound may be as hereinbefore described.

The method of manufacturing may include a further step of packaging the mixture of carrier and zinc compound while said mixture is still in liquid form.

In a further aspect of the invention there is provided a mechanical contraceptive device which defines barrier surfaces adapted for contact with genital organs, wherein at least a portion of one of the barrier surfaces is coated with a topical composition or pharmaceutical formulation according to the invention.

The invention further provides an article of manufacture which includes a mechanical contraceptive device as described herein; and a sealed package containing said contraceptive device.

According to yet another aspect of the invention there is provided a method of manufacturing a mechanical contraceptive device defining barrier surfaces adapted for contact with genital organs, the method including coating at least a portion of one of the barrier surfaces with a topical composition or pharmaceutical formulation according to the invention.

The contraceptive device may be selected from the group consisting of a male condom and a female condom.

### DETAILED DESCRIPTION OF EMBODIMENTS OF THE INVENTION

Embodiments of the invention will now be described by way of non-limiting example.

A specific example of a topical composition according to the invention is formulated as follows (all percentages by weight):

40% zinc oxide, 20% petrolatum, 9% lanolin oil, 13.5% mineral oil, 10% emulsifying wax, 3.87% beeswax, 1% glyceryl caprylate, 1% glyceryl laurate, 0.5% dioxide and/or diol (phenoxyethanol, caprylyl glycol and hexylene glycol) and 1% iron oxide for colouring.

An important feature of the invention is the high concentration of non-water soluble or sparingly water-soluble zinc compounds (zinc oxide in the above example), added to an emulsifier or carrier agent that is non-irritating to the skin and non-toxic.

Improved performance is understood to occur with higher concentrations.

Preferred compositions and formulae according to this invention are anhydrous, which means there is no water content and as such they are water resistant or even “water proof”.

In selected embodiments of the invention, zinc oxide in the form of commercially available nanopowder is advantageously used. This is made up of nanoparticles having individual sizes not exceeding 100 nm and a surface area typically ranging from 15-25 m²/g. (Normal zinc oxide used in formulations has a particle size much greater than 100 nm.) Special processes known in the art are used to reduce the particle size to the nanoparticle range. These processes also affect the colouration of the zinc oxide, for example colour may be removed. When zinc oxide in the form of nanoparticles is used to make up the topical composition of the invention, the usual colouration of zinc oxide can be changed or reduced in opacity so that the composition when applied to the skin is substantially clear.

In some embodiments of the invention, the topical composition may include a colourant that can impart to it a colour selected to match a particular human skin-tone. For example, where a user has a darker skin-tone (like brown) the user may experience some self-consciousness or embarrassment if they are expected to apply to their body a composition having a clearly contrasting colour, such as the bright white colour often associated with zinc oxide. Accordingly, different hues and shades of colourants may be included in the compositions to appeal to users having different skin tones. For example, to cater to the user mentioned in the case above, a brown colourant may be included in the composition.

It will be understood by one skilled in the art that many different formulations of topical compositions may be made up and will fall within the scope of the present invention.

The topical compositions of the invention may be provided as creams, ointments, lotions or the like, and are intended for prophylactic, inhibitory or preventive use to limit transmission of microorganisms responsible for certain STDs (“STDs”), for example, the Human Immunodeficiency Virus (“HIV”), Herpes and the Human Papilloma Virus (“HPV”), between sexual partners.

In use, a topical composition according to the invention is applied to or coated over one or more genital surfaces. For example, the composition can be applied to a man's penis and genitals, or to part of the interior or exterior surfaces of a vagina. The composition is typically so applied prior to and/or after sexual contact with a partner.

For additional protection the composition may be used in conjunction with a male or female condom.

The composition has been developed to inhibit STD microorganisms by microbicidal action, and as a barrier to restrict the entry of such microorganisms into a person's blood supply.

The following is a specific example of a method of manufacturing a topical composition according to the invention. The steps involved include:

heating components selected from petrolatum, lanolin oil, mineral oil, emulsifying wax, beeswax, glyceryl caprylate and glyceryl laurate to 70-75 degrees Celsius;

mixing said components until a substantially homogenous mixture is formed;

adding a zinc compound, preferably zinc oxide, using a high shear mixing device while maintaining the temperature of 70-75 degrees Celsius;

adding iron oxide using a high shear mixing device until the formulation reaches a smooth consistency;

allowing the formulation to cool to 55-60 degrees Celsius while maintaining agitation (e.g. by standard mixing);

adding a dioxide and/or diol (phenoxyethanol, caprylyl glycol and hexylene glycol);

cooling the mixture to 35-40 degrees Celsius; and packaging the mixture while still in liquid or semi-solid form, thereby to form said topical composition.

Numerous formulations of topical compositions were created and tested by the Inventor during research and development leading up to the present invention, before the Inventor settled on a preferred formulation which includes 40% zinc oxide along with a carrier and components as hereinbefore described.

The preferred formulation of the composition was subjected to a series of tests.

The first test was a USP 51 test where contaminants were added to the formulation to test to see if bacteria or viruses would grow or could be cultivated. The test showed that even after 6 weeks, insignificant growth was detected.

Further tests were carried out by ATS Laboratories of Minnesota, as follows:

The formulation was mixed with the Herpes Simplex 2 virus and measurements were taken to determine whether or not the virus was killed by the formulation, and the time frame involved. This set of tests established that 99.4% of the virus was killed within 60 seconds of exposure to the formulation.

The above methodology was also used to carry out another set of tests, this time using the HIV virus, and resulted in 99.9% of the HIV virus being killed within 60 seconds.

The next test conducted by ATS Laboratories was a barrier test whereby the formulation was applied to a 0.45 μm membrane filter. A test virus, of a similar molecular size as the HIV and Herpes virus, was applied to the formulation to see if the formulation functioned as a barrier to prevent the viruses from penetrating the formulation thus having the potential to penetrate the skin. The test showed that even over a 3-hour exposure, undetected numbers of the virus could penetrate the formulation thus confirming that the formulation set up a skin protecting barrier for an extended period of time.

A final set of tests were conducted in a qualitative manner and involved the use of the preferred formulation during sexual acts to test for side effects, irritation, discomfort and negative interactions. The formulation was found, at times, to mix to a small degree with mucous and discolour the mucous to a minimal degree. No significant irritation, discomfort or negative side effects were observed.

The topical compositions described herein may have certain advantages. For example, the compositions described provide high ratios of zinc oxide relative to other components in the composition, and high ratios of zinc oxide compared with other topical compositions available. The Inventor reached the insight that a composition containing low concentrations of zinc oxide creates an inadequate barrier, insufficient to withstand the friction and mucous generated during sexual acts.

The topical compositions described herein have advantages over the teachings of the '675 and '053 patents mentioned previously. Those patents teach the use of water soluble zinc salts, which can be irritating to the skin and can only be used in small concentrations. The topical compositions described herein make use of zinc compounds which are only sparingly soluble in water.

Furthermore, the above patents refer to the use of the zinc salts in a water-based formulation, such as an aqueous mixture or an aqueous emulsion. Such a formulation may lack sufficient viscosity to set up a barrier capable of remaining intact during sexual acts and thus protecting users during the rigours of sexual friction and the generation of mucous in quantity. Selected embodiments of the topical compositions described herein may address the above disadvantage by their inclusion, in relatively high ratios, of a barrier substance such as petrolatum.

1. A topical composition for use in the control of sexually transmitted diseases, wherein:
between 25% and 50% by weight of said composition is comprised of at least one pharmaceutically acceptable zinc compound selected from the group consisting of non water-soluble and sparingly water-soluble zinc compounds; and
said composition further includes at least one pharmaceutically acceptable carrier within which the at least one zinc compound is dispersed.

2. The topical composition of claim 1, in which the zinc compound is zinc oxide.

3. The topical composition of claim 2, in which the zinc compound is present in the topical composition in a ratio ranging from 38% to 42% inclusive by weight.

4. The topical composition of claim 1, further comprising a barrier substance present in a ratio ranging from 1% to 30% inclusive by weight.

5. The topical composition of claim 4, in which the barrier substance comprises a hydrocarbon mixture.

6. The topical composition of claim 1, which includes a colourant adapted to impart to the composition a colour selected to match a predetermined human skin-tone.

7. The topical composition of claim 1, comprising a plurality of components selected from the group consisting of the following, present in the ranges stated (by weight):
either one of zinc oxide and a zinc salt (between 25% and 50%);
petrolatum (1% to 30% inclusive);
lanolin oil (1% to 20% inclusive);
mineral oil (1% to 20% inclusive);
emulsifying agent (5% to 15% inclusive);
beeswax (2% to 8% inclusive);
glyceryl caprylate (1% to 2% inclusive);
glyceryl laurate (0.1% to 2% inclusive);
a component selected from dioxides, diols and mixtures of these (0.1% to 2% inclusive); and
iron oxide (0.1% to 2% inclusive).

8. A topical composition for use in the control of sexually transmitted diseases, wherein:
from 1% to 60% inclusive by weight of said composition is comprised of at least one pharmaceutically acceptable zinc compound selected from the group consisting of non water-soluble and sparingly water-soluble zinc compounds having particles the majority of which have a maximum dimension not exceeding 100 nanometers; and
said composition further includes at least one pharmaceutically acceptable carrier within which the at least one zinc compound is dispersed.

9. The topical composition of claim 8, in which the zinc compound is zinc oxide.

10. The topical composition of claim 9, in which the zinc compound is present in the topical composition in a ratio ranging from 38% to 42% inclusive by weight.

11. The topical composition of claim 8, further comprising a barrier substance present in a ratio ranging from 1% to 30% inclusive by weight.

12. The topical composition of claim 11, in which the barrier substance comprises a hydrocarbon mixture.

13. The topical composition of claim 8, which includes a colourant adapted to impart to the composition a colour selected to match a predetermined human skin-tone.

14. The topical composition of claim 8, comprising a plurality of components selected from the group consisting of the following, present in the ranges stated (by weight):
either one of zinc oxide and a zinc salt (between 25% and 50%);
petrolatum (1% to 30% inclusive);
lanolin oil (1% to 20% inclusive);
mineral oil (1% to 20% inclusive);
emulsifying agent (5% to 15% inclusive);
beeswax (2% to 8% inclusive);
glyceryl caprylate (1% to 2% inclusive);
glyceryl laurate (0.1% to 2% inclusive);
a component selected from dioxides, diols and mixtures of these (0.1% to 2% inclusive); and
iron oxide (0.1% to 2% inclusive).

15. A method of manufacturing a topical composition for prophylaxis against sexually transmitted diseases, which includes the steps of
heating at least one pharmaceutically acceptable carrier to a liquid form; and
mixing with said carrier at least one pharmaceutically acceptable zinc compound selected from the group consisting of non water-soluble and sparingly water-soluble zinc compounds, in an amount calculated to result in a ratio of the at least one zinc compound, in the manufactured composition, ranging between 25% and 50% by weight.

16. The method of claim 15, further comprising a further step of packaging the mixture of carrier and zinc compound while said mixture is still in liquid form.

17. A mechanical contraceptive device, comprising:
a body defining barrier surfaces adapted for contact with genital organs, wherein at least a portion of one of the barrier surfaces is coated with a topical composition comprising:
between 25% and 50% by weight of said composition is comprised of at least one pharmaceutically acceptable zinc compound selected from the group consisting of non water-soluble and sparingly water-soluble zinc compounds; and
said composition further includes at least one pharmaceutically acceptable carrier within which the at least one zinc compound is dispersed.

18. The mechanical contraceptive device of claim 17, comprising either one of a male condom and a female condom.

19. A mechanical contraceptive device, comprising:
a body defining barrier surfaces adapted for contact with genital organs, wherein at least a portion of one of the barrier surfaces is coated with a topical composition comprising:
from 1% to 60% inclusive by weight of said composition is comprised of at least one pharmaceutically acceptable zinc compound selected from the group consisting of non water-soluble and sparingly water-soluble zinc compounds having particles the majority of which have a maximum dimension not exceeding 100 nanometers; and
said composition further includes at least one pharmaceutically acceptable carrier within which the at least one zinc compound is dispersed.

20. The mechanical contraceptive device of claim 19, comprising either one of a male condom and a female condom.
